(19) United States
(12) Patent Application Publication
(10) Pub. No.: US 2004/0260329 A1
(43) Pub. Date: Dec. 23, 2004

(54) Catheter and guide wire exchange system with decoupled guide member

(76) Inventors: Richard Gribbons, Athenry (IE); John MacNamara, Salthill (IE); Niall Duffy, Tuam (IE); Ashish Varma, Kinvara (IE); Gerry Clarke, Moycullen (IE); Mark Casley, Taylors Hill (IE); Noel Coyle, Craughwell (IE); David Quinn, Salthill (IE); Kevin Boyle, Renmore (IE); Kevin Treacy, Athenry (IE); Patrick Duane, Taylors Hill (IE); Rodney Bell, Doughiska (IE)

Correspondence Address: MEDTRONIC VASCULAR, INC., IP LEGAL DEPARTMENT, 3576 UNOCAL PLACE, SANTA ROSA, CA, 95403, US

(21) Appl. No.: 10/722,191

(22) Filed: Nov. 24, 2003

Related U.S. Application Data
(60) Provisional application No. 60/479,695, filed on Jun. 19, 2003.

(51) Int. Cl. A61M 29/00

(52) U.S. Cl. 606/194

(57) ABSTRACT

A catheter and guide wire exchange system including a catheter having a guide wire lumen with a guide way extending along the length of a stiffened proximal shaft portion, and a guide member slidably disposed about the proximal shaft for directing a guide wire into or out of the guide way and the guide wire lumen. The proximal shaft may be slid through the guide member so that the guide wire is contained within the guide wire lumen distal to the guide member and with the guide wire and catheter being separated proximal of the guide member. The guide member has a decoupled body, that allows it to be rotated without affecting the guidewire direction through the guidemember.

### FIELD OF THE INVENTION

The present invention relates to catheters used in the vascular system and more particularly to a system for facilitating exchange of such catheters and guide wires, and for using such catheters and guide wires to access selected sites within a patient.

### BACKGROUND OF THE INVENTION

Catheters are inserted to various locations within a patient for a wide variety of purposes and medical procedures. For example only, one type of catheter is used in percutaneous catheter intervention (PCI) for the treatment of a vascular constriction termed a stenosis. In this instance, the catheter has a distally mounted balloon that can be placed, in a deflated condition, within the stenosis, and then inflated to dilate the narrowed lumen of the blood vessel. Such balloon dilation therapy is generally named percutaneous transluminal angioplasty (PTA). The designation PTCA, for percutaneous transluminal coronary angioplasty, is used when the treatment is more specifically employed in vessels of the heart. PTCA is used to open coronary arteries that have been occluded by a build-up of cholesterol fats or atherosclerotic plaque. The balloon at the distal end of the catheter is inflated, causing the site of the stenosis to widen.

The dilation of the occlusion, however, can form flaps, fissures and dissections, which may result in reclosure of the dilated vessel or even perforations in the vessel wall. Implantation of a stent can provide support for such flaps and dissections and thereby prevent reclosure of the vessel or provide a patch repair for a perforated vessel wall until corrective surgery can be performed. A stent is typically a cylindrically shaped device formed from wire(s) or a metal tube and is intended to act as a permanent prosthesis. A stent is deployed in a body lumen from a radially compressed configuration into a radially expanded configuration that allows it to contact and support a body lumen. A stent can be implanted during an angioplasty procedure by using a balloon catheter bearing a compressed stent that has been loaded onto the balloon. The stent radially expands as the balloon is inflated, forcing the stent into contact with the body lumen, thereby forming a supporting relationship with the lumen walls. Alternatively, self-expanding stents may be deployed with a sheath-based delivery catheter. Deployment is effected after the stent has been introduced percutaneously, transported transluminally and positioned at a desired location by the delivery catheter. In addition to angioplasty and stenting procedures, other therapeutic procedures require use of a delivery catheter, such as drug delivery, filters, occlusion devices, diagnostic devices and radiation treatment.

Typically, the placement of such therapeutic delivery catheters involves the use of a guide wire, which may be inserted into the patient's vasculature through the skin, and advanced to the location of the treatment site. The delivery catheter, which has a lumen adapted to receive the guide wire, then is advanced over the guide wire. Alternatively, the guide wire and the delivery catheter may be advanced together, with the guide wire protruding from the distal end of the delivery catheter. In either case, the guide wire serves to guide the delivery catheter to the location to be treated.

There are four general types of catheters: “over-the-wire” (OTW) catheters, Multi-Exchange catheters (MX) such as disclosed in U.S. Pat. No. 4,998,356 (Crittenden, et al.) and co-pending applications U.S. Ser. No. 10/116,234, Ser. No. 10/251,578, filed Sep. 20, 2003 and Ser. No. 10/251,477, filed Sep. 20, 2003, which are incorporated in their entirety herein by reference “rapid exchange” catheters and “fixed wire” or “a balloon on a wire” catheters. OTW and rapid exchange catheters require use of a guide wire separate from the catheter while a fixed wire or balloon on a wire catheter has an integral guide wire. An OTW catheter comprises a guide wire lumen that extends the entire length of the catheter. The guide wire is disposed entirely within the catheter guide wire lumen except for distal and proximal portions of the guide wire, which extend beyond the distal and proximal ends of the catheter respectively. An MX catheter has an over-the-wire configuration while the catheter is within the patient's body. Thus, the guide wire is disposed entirely within the catheter guide wire lumen, except for the distal and proximal portion of the guide wire, which extend beyond the distal and proximal ends of the catheter respectively when it is fully inserted into the patient.

OTW and MX catheters have many advantages traceable to the presence of the full length guide wire lumen, such as good stiffness and pushabilty for readily advancing the catheter through the tortuous vasculature and across tight stenosis. The full-length guide wire lumen permits removal and replacement of a guide wire in an indwelling catheter, as may be required to alter the shape of the guide wire tip. It is also sometimes desirable to exchange one guide wire for another guide wire having a different stiffness. For example, a relatively soft, or flexible guide wire may prove to be suitable for guiding a PTCA catheter through a particular tortuous anatomy, whereas following up with a stent delivery catheter through the same vasculature region may require a guide wire that is relatively stiffer.

Traditional over-the-wire catheters do suffer some shortcomings, however. For example, it often becomes necessary, in the performance of a PCI, to exchange one indwelling catheter for another catheter. In order to maintain a guide wire in position while withdrawing the catheter, the guide wire must be gripped at its proximal end to prevent it from being pulled out of the blood vessel with the catheter. For example, a PTCA catheter, which may typically be on the order of 135 centimeters long, is longer than the proximal portion of the standard guide wire that protrudes out of patient. Therefore, exchanging an over-the-wire PTCA catheter requires an exchange guide wire of about 300 centimeters long, whereas a standard guide wire is about 165 centimeters long.

In one type of over-the-wire catheter exchange, the standard length guide wire first is removed from the lumen of the indwelling catheter. Then, the longer exchange guide wire is passed through the catheter to replace the original wire. Next, while holding the exchange guide wire by its proximal end to control its position in the patient, the catheter is withdrawn proximally from the blood vessel over the exchange guide wire. After the first catheter has been removed, the next OTW catheter is threaded onto the proximal end of the exchange guide wire and is advanced along the exchange guide wire, through the guiding catheter, and into the patient's blood vessels until the distal end of the catheter is at the desired location. The exchange guide wire may be left in place or it may be exchanged for a shorter, conventional-length guide wire. In an alternative type of catheter exchange procedure, the length of the initial guide wire may be extended by way of a guide wire extension apparatus. Regardless of which exchange process is used, the very long exchange guide wire is awkward to handle, thus requiring at least two operators to perform the procedure.

A balloon catheter capable of both very fast exchange and simple guidewire and catheter exchange is particularly advantageous. A catheter designed to address this need sold by Medtronic Vascular of Santa Rosa, Calif. under the trademarks MULTI-EXCHANGE, ZIPPER MX, ZIPPER and/or MX is disclosed in U.S. Pat. No. 4,988,356 (Crittenden et al.) and pending U.S. application Ser. No. 10/116,234 filed Apr. 4, 2003; Ser. No. 10/251,578, filed Sep. 20, 2003 and Ser. No. 10/251,477, filed Sep. 20, 2003, which are incorporated in their entirety herein by reference. A MX catheter includes a catheter shaft having a cut that extends longitudinally between the proximal end and the distal end of the catheter and that extends radially from the catheter shaft outer surface to the guide wire lumen. A guide member coupled to the catheter shaft functions to temporarily open the cut such that the guide wire may extend transversely into or out of the cut at any location along its length. By moving the proximal shaft through the guide member, the effective over-the-wire length of the MX catheter is adjustable.

When using the MX catheter, the guide wire is maneuvered through the patient's vascular system such that the distal end of the guide wire is positioned across the treatment site. With the guide member positioned near the distal end of the catheter, the proximal end of the guide wire is threaded into the guide wire lumen opening at the distal end of the catheter and through the guide member such that the proximal end of the guide wire protrudes out the proximal end of the guide member. By securing the guide member and the proximal end of the guide wire in a fixed position, the catheter may then be delivered over the guide wire by advancing the catheter toward the guide member. In doing so, the catheter advances through the guide member such that the guide wire lumen envelops the guide wire as the catheter is advanced into the patient's vasculature. In a PTCA embodiment, the MX catheter may be advanced over the guide wire in this manner until the distal end of the catheter having the dilatation balloon is positioned within the stenosis and essentially the entire length of the guide wire is encompassed within the guide wire lumen.

Furthermore, the indwelling MX catheter may be exchanged with another catheter by reversing the operation described above. To this end, the indwelling catheter may be removed by withdrawing the proximal end of the catheter from the patient while holding the proximal end of the guide wire and the guide member in a fixed position. When the catheter has been withdrawn to the point where the distal end of the cut has reached the guide member, the distal portion of the catheter over the guide wire is of a sufficiently short length that the catheter may be drawn over the proximal end of the guide wire without releasing control of the guide wire or disturbing its position within the patient. After the catheter has been removed, another MX catheter may be threaded onto the guide wire and advanced over the guide wire in the same manner described above with regard to the MX catheter. The MX catheter not only permits catheter exchange without the use of the very long exchange guide wire and without requiring withdrawal of the initially placed guide wire, but it also overcomes many of the other difficulties discussed in association with rapid exchange catheters described below.

Rapid exchange catheters developed in an attempt to eliminate the need for a guide wire extension or exchange wires. Catheters of this type are formed so that the guide wire is located outside of the catheter except for a short guide wire lumen that extends within only a comparatively short distal segment of the catheter. The rapid exchange catheter's proximal exit port for the guide wire is typically located about 5 cm (2.0 in) to 30 cm (11.8 in) proximal to the catheter's distal end. In use, the guide wire is placed initially in the patient's vascular system. The distal segment of the rapid exchange catheter then is threaded onto the wire. The catheter can be advanced alongside the guide wire with its distal segment being attached to and guided along the guide wire. The rapid exchange catheter can be removed and exchanged for another rapid exchange catheter without the use of a very long exchange guide wire and without requiring withdrawal of the initially placed guide wire.

A difficulty associated with rapid exchange catheters is that it is not possible to exchange guide wires in an indwelling rapid exchange catheter, as can be done advantageously with OTW catheters. A guide wire can be withdrawn, sometimes unintentionally, from the proximal guide wire port, thus derailing an indwelling rapid exchange catheter. However, neither the first guide wire, nor a replacement guide wire, can be directed back into the catheter's proximal guide wire port, which is hidden remotely in the guiding catheter within the patient.

Guide wires are commonly back loaded into the delivery catheter. In this operation, the guide wire proximal end is inserted into the distal tip of the catheter. It is pushed through the catheter until it extends out of the proximal guide wire exit. In a traditional over-the-wire catheter the proximal guide wire exit is the proximal end of the catheter through its inflation luer. The rapid exchange proximal guide wire exit is the termination of the short guide wire tube a few centimeters or typically 25 centimeters beyond the distal tip of the catheter. In the MX catheter, the proximal guide wire exit is through the guide member positioned on the proximal shaft of the catheter. As an alternative to back loading a guide wire into the delivery system, a guide wire may also be front-loaded. In a front-loading operation, the distal tip of the guide wire is inserted into the guide wire lumen on the proximal shaft and pushed through until it exits the distal tip of the delivery catheter. A front-loading operation is possible with OTW and MX catheters if the guide wire will be exchanged during procedures. A front loading operation is not used with a rapid exchange catheter since the guide wire cannot be exchanged while the catheter is inserted into the patient. With a rapid exchange catheter, the insertion of the distal tip into the proximal end of the guide wire lumen is pure chance due to the fact that the proximal end is typically 125 centimeters from the exit location of the catheter from the patient at the femoral artery in the groin.

The guide member of the MX catheter is used for both advancement of the catheter into the patient and for exchanging the guide wire during the procedure without removing the catheter. In order to further optimize handling of the catheter, it is desirable to permit the user the flexibility to rotate the proximal shaft without affecting its placement with respect to the guide member and entry of the guidewire into the proximal shaft. In current MX catheters, the practitioner must ensure that the proximal shaft remains aligned with the guide member passageway. Severe torquing of the proximal shaft may result in increased force necessary to introduce the proximal shaft through the guide member passageway to engage the keel. Thus, the present invention is directed towards various embodiments of the guide member that optimize the versatility of the dual function of the guide member while permitting the user more flexibility in handling. Additionally it is desirable to decrease the profile of the MX catheter to allow for more room in guide catheter to enable more access to distal sites and allow a greater amount of dye to be flushed down the guide catheter for visualization. Thus there is a need for a smaller keel and smaller profile.

### SUMMARY OF THE INVENTION

The present invention is a guide member for an MX catheter and guide wire exchange system. The MX catheter and guide wire exchange system comprises an elongate flexible catheter having proximal and distal ends and first and second lumens extending there through. The first lumen is open at the shaft distal end and is sized and shaped to receive a guide wire. The second lumen is sized and shaped to receive inflation fluid therethrough. The catheter has a proximal shaft that may be either bitumen or tri-lumen. The distal shaft is preferably coaxial. The guide member is mounted on the catheter proximal shaft and its keel is received in a guide way formed from a longitudinal cut in a catheter proximal shaft to enable transverse access to the guide wire lumen. The guide way extends along a major portion of the length of the proximal shaft from a location adjacent to the proximal end of the catheter to a location proximal of the proximal shaft distal end. An enlarged stop is located on the exterior of the proximal shaft distal end. The guide member cannot travel distally past the stop. A balloon is mounted about catheter distal segment, with the balloon being in fluid communication with the second lumen.

The guide member has a catheter passageway that extends longitudinally through the guide member and a guide wire passageway for slidably receiving a guide wire therethrough. The guide member keel cooperates with the guide way to assist in merging the guide wire into the first lumen as the catheter shaft is moved through the catheter passageway. Conversely, the guide member can be used for separating the guide wire and catheter by guiding the guide wire out of the guide wire lumen through the guide way. The guide member contains an outer member that rotates freely around the guide member positioned on the catheter shaft. Rotation of the outer member does not affect the position of the guide member keel with respect to the longitudinal cut.

### BRIEF DESCRIPTION OF THE DRAWINGS

These and other features, aspects and advantages of the present invention will become better understood with reference to the following description, appended claims, and accompanying drawings where:

FIG. 1 is an illustration of a MX catheter and guide wire in an assembled configuration;

FIG. 1A is a cross-section taken along line A-A of FIG. 1;

FIG. 1B is a cross-section taken along line B-B of FIG. 1;

FIG. 1C is a cross-section taken along line C-C of FIG. 1;

FIG. 1D is a cross section taken along line D-D of FIG. 1;

FIG. 2 is a longitudinal sectional view of a distal section of a bi-lumen proximal shaft embodiment of the present invention having an oval configuration incorporating an alternative stop embodiment;

FIG. 3 is an end view of a bitumen proximal shaft embodiment of the present invention having a circular configuration

FIG. 4 is an end view of a tri-lumen proximal shaft embodiment of the present invention having a triangular configuration;

FIG. 5 is a perspective end view of a tri-lumen proximal shaft embodiment of the present invention having a shamrock configuration;

FIG. 6 is a longitudinal cross section view of the intersection of the guide member and proximal shaft of a MX catheter;

FIG. 7 is a perspective elevational view of the guide member of the present invention from the proximal end;

FIG. 8 is a perspective elevational view of the guide member of the present invention from the distal end;

FIG. 9 is a longitudinal cross section view of the main body of the guide member of the present invention having an alternative clipping mechanism;

FIG. 10 is a perspective elevational view of the main body of the guide member of the present invention;

FIG. 11 is cross section view of the main body of the present invention;

FIG. 12 is a perspective elevational view of an alternative embodiment of the main body of the present invention;

FIG. 13 is a perspective elevational view of the keel of the present invention; and

FIG. 14 is a cross section view showing the keel engaging the proximal shaft of present invention.

### DETAILED DESCRIPTION OF THE PREFERRED EMBODIMENT

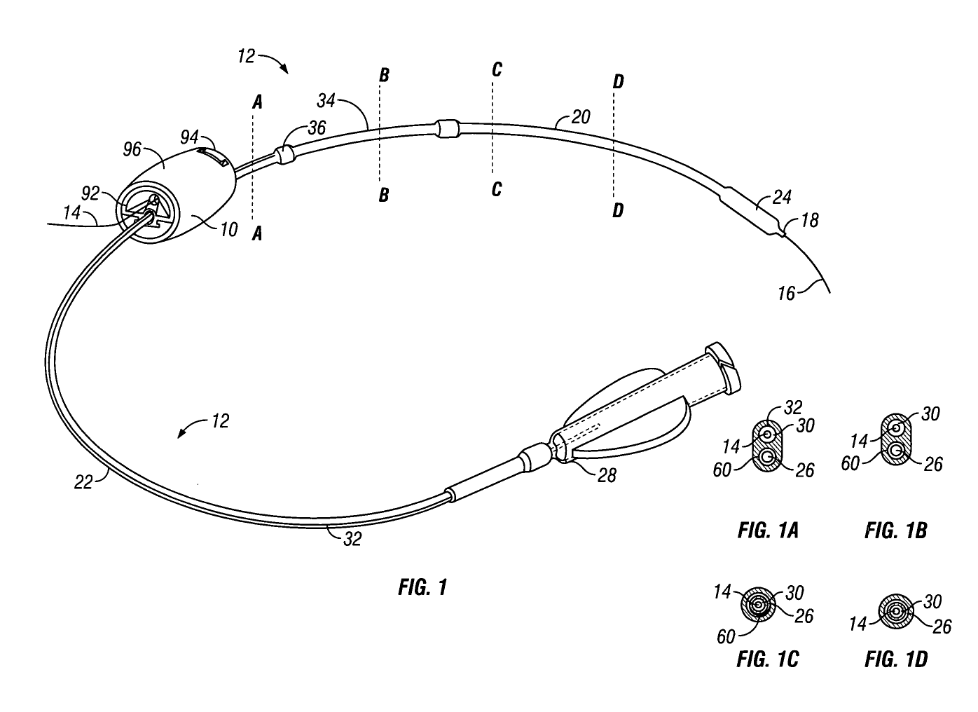

The present invention is a guide member 10 for MX catheter 12 shown in FIGS. 1 and 1A-1D with guide wire 14 illustrated as extending through guide member 10 and catheter 12. Guide member 10 serves as a juncture in which the catheter 12 and guide wire 14 may be merged or separated so that the portion of guide wire 14 which extends proximally of guide member 10 (to the left as seen in FIG. 1) is separated from catheter 12 and the portion of guide wire 14 which is located distally of guide member 12 (to the right as seen in FIG. 1) is contained and housed within catheter 12 except for distal end 16 of guide wire 14 which may protrude distally out of catheter distal end 18.

Catheter 12 includes an elongate, flexible, cylindrical main body having a distal shaft 20 and a proximal shaft 22. In the embodiment shown in FIG. 1, catheter 12 is a delivery catheter, such as for PTCA or stent delivery, having balloon 24 mounted around the catheter body near catheter distal end 18. Balloon 24 may be inflated and deflated through inflation lumen 26 formed through the body of the catheter 12. Inflation lumen 26 extends from the proximal end of catheter 12, where it communicates with fitting 28 and extends the length of catheter 12, terminating in communication with the interior of balloon 24. Fitting 28 may be connected to a suitable source of pressurized fluid or a partial vacuum (not shown) to inflate or deflate balloon 24. Catheter 12 includes another lumen, indicated at 30, which is intended to receive guide wire 14. Guide wire lumen 30 extends the full length of catheter 12, terminating at distal end 18 and proximal fitting 28. A longitudinal cut extends into the guidewire lumen along most of the length of proximal shaft 22 to form guideway 32. The distal section 34 of proximal shaft does not contain guideway 32 as seen in FIGS. 1 and 1B.

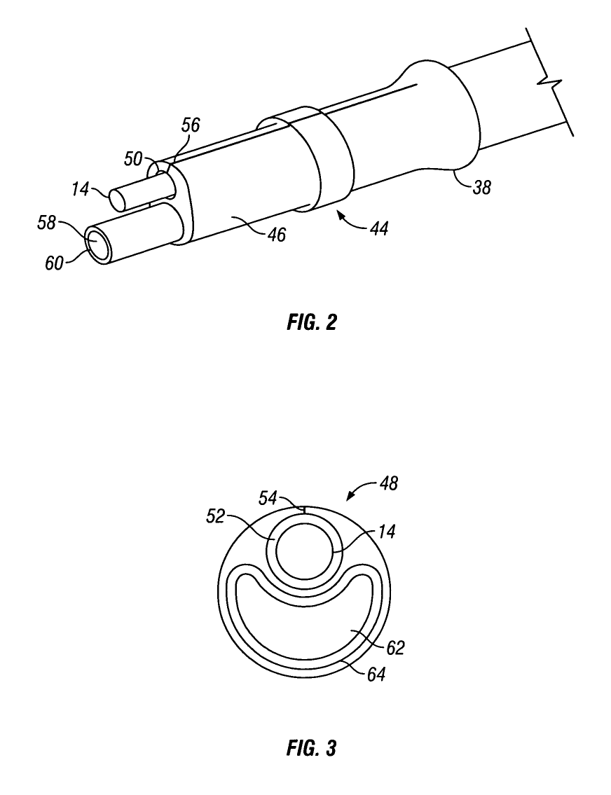

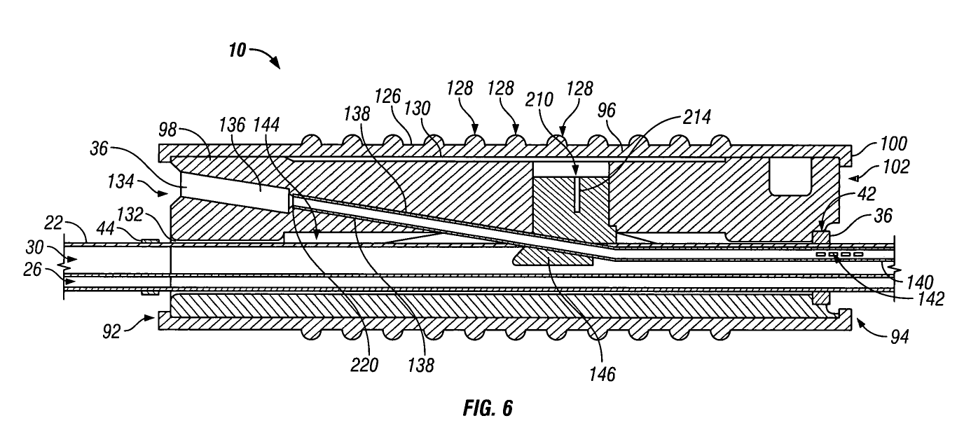

Proximal shaft 22 preferably contains stop 36 adjacent its distal section 34. Stop 36 may be an enlarged section of proximal shaft 22 that prevents guide member 10 from being forced onto distal shaft 20. Stop 36 may be annular or a series of raised areas radially spaced around proximal shaft 22. Stop 36 may act as a wall against which guide member 10 abuts, as shown in FIG. 1, or an angled ramp 38, as shown in FIG. 2, against which guide member 10 wedges. Lastly, as shown in FIG. 6, stop 36 may create an interference fit with docking area 42 on guide member 10. A smaller raised area may also be located on proximal shaft 22 to act as a speed bump as shown in FIG. 2. Like stop 36, speed bump 44 is an enlarged section of proximal shaft. However, speed bump 44 is small enough to allow proximal shaft to over ride it as proximal shaft 22 passes through guide member 10. Speed bump 44 is spaced proximally from stop 36 such that guide member 10 is positioned between stop 36 and speed bump 44 when guide member 10 is in its most distal position on proximal shaft 22. Speed bump 44 will advise the practitioner when stop 36 is near the guide member 10. It also will hold guide member 10 in its distal position during a backloading operation as will be described in greater detail below.

Distal shaft 20 is preferably coaxial as shown in FIGS. 1C and 1D and contains inflation lumen 26 and guide wire lumen 30. Proximal shaft 22 may be a bi-lumen shaft or a tri-lumen shaft. Co-pending patent application titled CATHETER AND GUIDE WIRE EXCHANGE SYSTEM WITH IMPROVED PROXIMAL SHAFT AND TRANSITION SECTION and filed concurrently with this application describes various proximal shaft arrangements for MX catheters and is incorporated herein by reference in its entirety. The bitumen shaft may be oval or circular as shown by proximal shafts 46 and 48 in FIGS. 2 and 3. Proximal shafts 46 and 48 each have guidewire lumens 50 and 52 that are accessible though guideways 54 and 56 located along the proximal shaft length as in the manner shown in FIG. 1. Inflation lumen 58 of proximal shaft 46 runs side by side along the length of proximal shaft 46 with guide wire lumen 50 and is preferably supported by a stiffening member 60, such as a hypotube. Inflation lumen 62 of shaft 42 is crescent shaped and also contains a stiffening member 64, such as a crescent shaped hypotube. Stiffening members 60 and 64 provide stiffness for force transmission along the length of the catheter 12. They may further include a transition section at their distal sections to ease the transition from the stiffer proximal shaft to the flexible distal shaft and avoid shaft kinking at the proximal shaft 22 and distal shaft 20 junction. For example, hypotube 60 may be skived at its distal end, with the skived portion extending into the distal section as shown in FIG. 1C.

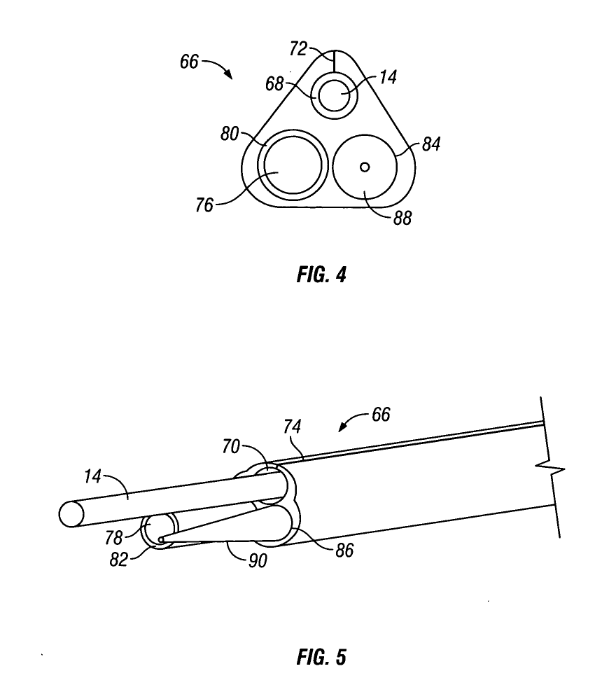

Turning now to FIGS. 4 and 5, trilumen shaft 66 may be generally circular (not shown), triangular or shamrock in its outer configuration, with the lumens preferably arranged in a triangular configuration as shown. Guide wire lumens 68 and 70 are accessible by guideways 72 and 74. Inflation lumens 76 and 78 preferably contain stiffening members 80 and 82, which may be hypotubes. Third lumens 84 and 86 contain stiffening wires 88 and 90. Stiffening wires 88 and 90 preferably taper from stiffer proximal shaft 22 towards more flexible distal shaft 24. Stiffening wires 88 and 90 preferably extend into distal shaft 20 to help transition catheter 12 from its stiffer proximal shaft 22 to its more flexible distal shaft 20. A stiffening wire is more resistant to kinking than the hypotube. Stiffening wires 88 and 90 may freely float within their lumens, be bonded at just their proximal end, be bonded at just their distal end or be bonded at their proximal and distal ends. Use of stiffening wires allow use of a thinner and smaller diameter hypotube in the inflation lumen since the tapered wire provides the stiffness and transition previously provided by just the hypotube. Thus the inflation lumen may be optimized for inflation deflation times as opposed to accommodating the hypotube with the appropriate stiffness for the catheter. Alternatively, the third lumen may be a second inflation lumen. In such an arrangement, a second hypotube or thinner stiffening wire may be used within the second inflation lumen.

Proximal shaft 22 is preferably comprised of polyethylene, but other suitable biomedical grade materials such as cross-linked PE, polyolefins, polyamides, blends of polyamides and polyolefins, fluoropolymers, polyesters, polyketones, polyimides, polysulphones, polyoxymethylens and compatibilisers based on polyolefins, included grafted polyolefins and other comparable materials may be used. A lubrication additive may also be used with any polymer and may include PE micro-powders, fluoropolymers, silicone based oils, fluoro-ether oils, molybdenum disulphide and polyethylene oxide. Additionally a reinforcing additive may be used such as nano-clays, graphite, carbon fibres, glass fibres and polymeric fibres. Distal shaft 20 is preferably made of a suitable polyethylene or polyolefin that readily bonds to proximal shaft 22.

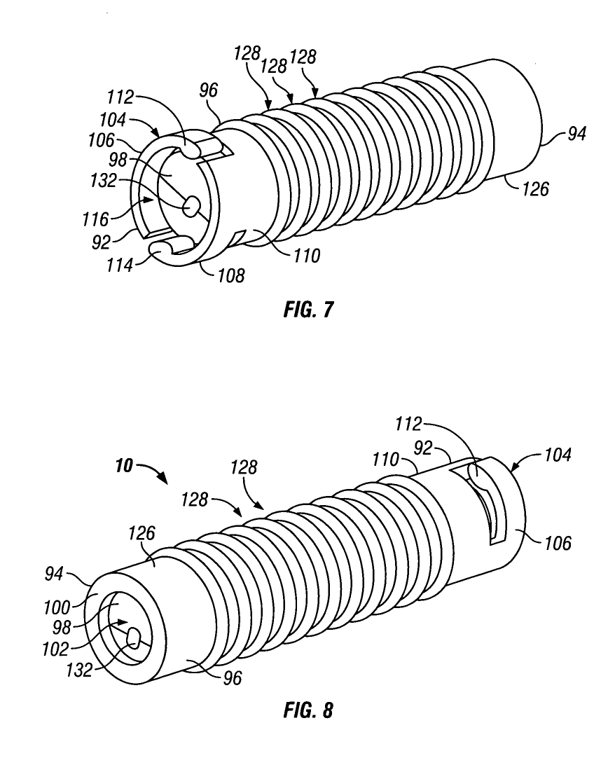

Guide member 10 surrounds proximal shaft 22 and has proximal and distal ends 92 and 94 as shown in FIGS. 1 and 6-12 and 14. Guide member 10 has an outer tubular member 96 that freely rotates around inner main body 98 and hence is decoupled from the inner main body 98. A stop consisting of an annular wall 100 extending into distal opening 102 of outer member 96 prevents main body 98 from slipping out of the outer member 96. A retaining clip mechanism 104 is positioned on proximal portion 92 of guide member 10. Retaining clip mechanism 104 consists of two arcuate arms 106 and 108 that form a portion of outer member wall 110 as seen in FIGS. 7 and 8. Each arm contains a tab, 112 and 114, that extends into proximal opening 116 of outer member 96 to prevent main body 98 from slipping out of outer member proximal opening 116. Arms 106 and 108 are opened up to remove tabs 112 and 114 from extending into proximal opening 116 to permit insertion of main body 98 during the assembly of guide member 10. While two tabs are shown positioned 180 degrees apart, a different number of tabs may be used, provided they are spaced sufficiently to prevent main body 98 from slipping out of outer member 96.

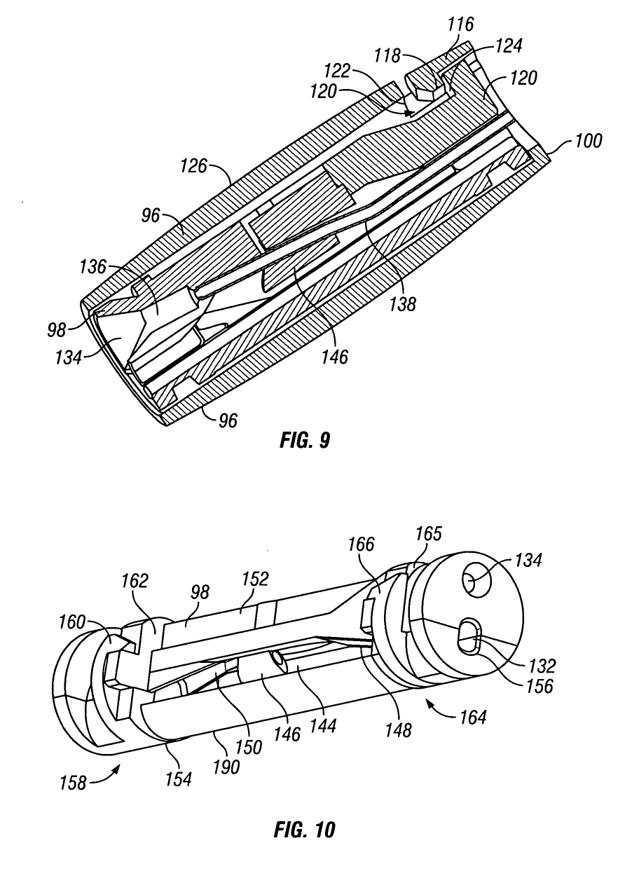

In an alternative retaining clip arrangement as shown in FIG. 9, retaining clip 116 contains tab 118 that extends into the space designated 120 formed by inner walls 122 and 124 of main body 98. Thus, when retaining clip 116 is in the closed position, tab 118 limits movement of main body 98 since tab 118 is captured between walls 122 and 124. While multiple tabs may be used, only one is necessary. Outer surface 126 may have a smooth surface as shown in FIG. 9 or a textured surface such as a surface with circumferential bosses designated 128 as shown in FIGS. 6, 7 and 8, to assist in grasping and manipulating guide member 10 as catheter shaft 22 is advanced through guide member 10. The inner surface 130 is smooth to facilitate rotation about main body 98. Furthermore, the materials selected may be chosen for their friction reduction and likewise a coating may be used on the inner surfaces to reduce friction.

Guide member main body 98 contains catheter passageway 132 extending longitudinally in a generally straight line from guide member proximal end 92 to guide member distal end 94. Guide wire passageway 134 extends distally from guide member end 92, through a passageway 136, into tube 138 and then into guide wire lumen 30.

Passageway 136 is configured to mate with a conventional wire introducer tool and further be tapered to aid in loading a conventional wire introducer tool. The length of tube 138 may vary however, it preferably extends through guide wire lumen 30 past the distal end 94 of guide member 10 as shown in FIG. 6 such that it will extend into distal shaft 20 when guide member 10 is positioned in its most distal position against stop 36. Any suitable length may be used, but it is preferably that it extends past the junction between proximal shaft 22 and distal shaft 20 to direct guide wire through the junction. Thirty-five millimeters is one such suitable length for distal portion 140 extending past guide member distal end 94. Apertures or cuts designated 142 may extend along the length of distal portion except for the very distal tip, such as the last 5 mm. The cuts may be a series of short cuts spaced along the length or may be a longer cut of 20-25 mm in length. Catheter passageway 132 is configured to slidingly receive the proximal shaft 22. Its shape preferably matches the proximal shaft shape and thus for proximal shaft 46 it is oval, for proximal shaft 48 it is circular, for proximal shaft 66 it is triangular or shamrock shaped. Catheter passageway 132 enlarges in a central area designated 144 into which keel 146 and guideway closing aids 148 and 150 extend minimizing frictional forces that may be result as the guideway engages keel 146. Catheter passageway 132 may further include docking area 42 for receiving stop 36 and shown in FIG. 6.

Turning now to FIGS. 10, 11 and 12, main body 98 is constructed from two parts, top 152 and base 154. Top 152 houses the guidewire passageway 134 along with keel 146, tube 138 and guideway closing aids 148 and 150. Base 154 forms a shaft support channel 156. Clipping mechanisms are used to secure top 152 and base 154. A snap fit assembly is preferable for ease of assembly while still assuring top 152 and base 154 are aligned when secured. In particular, as seen in FIG. 10 clipping mechanism 158 consists of arcuate arm 160 extending from base 154 that engages arm 162 formed on top 152. Clipping mechanism 164 operates in the same fashion with its arms 165 and 166. Four clipping mechanism ensure a secure fit and thus additional clipping mechanisms are located opposite clipping mechanisms 158 and 164. An alternative clipping arrangement is shown in FIG. 12. In this arrangement top 152 has arms 168, 170 and 172 that interlock with arms 174, 176 and 178 on base 154. A fourth clipping mechanism is located opposite interlocking arms 172 and 174.

Main body 98 contains arcuate surfaces 180, 182, 184 and 186 that form rotating surfaces tangent to rotating outer member 96. Support channel 156 formed along the bottom of catheter passageway 132 holds proximal shaft 22 under keel 146. Outer surface 190 is also in tangential rotating contact with outer member 96. Surface 190, along with surfaces 180, 182, 184 and 186 are smooth to reduce any frictional forces caused by rotating outer member 96 about main body 98. A clearance of approximately 0.05 mm exists between the rotating surfaces is preferable.

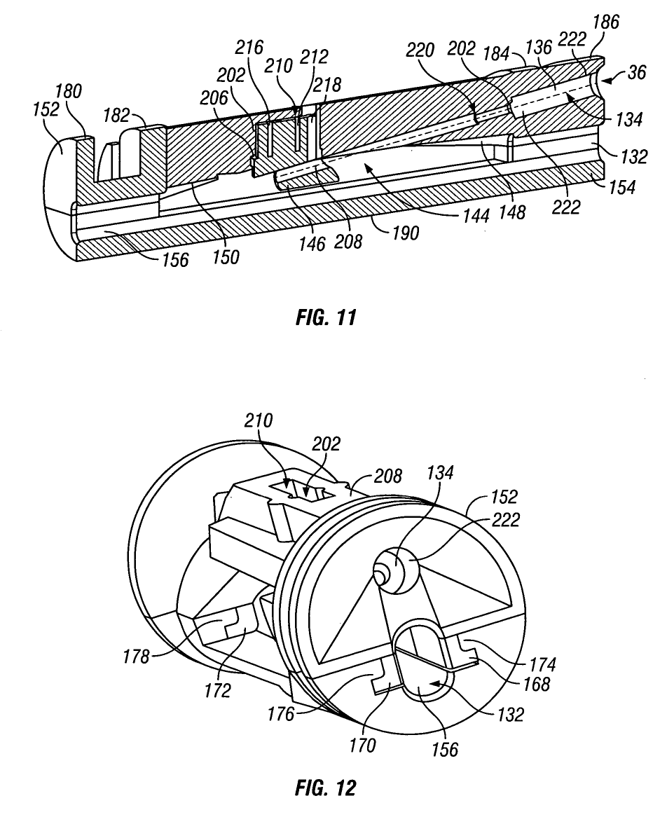

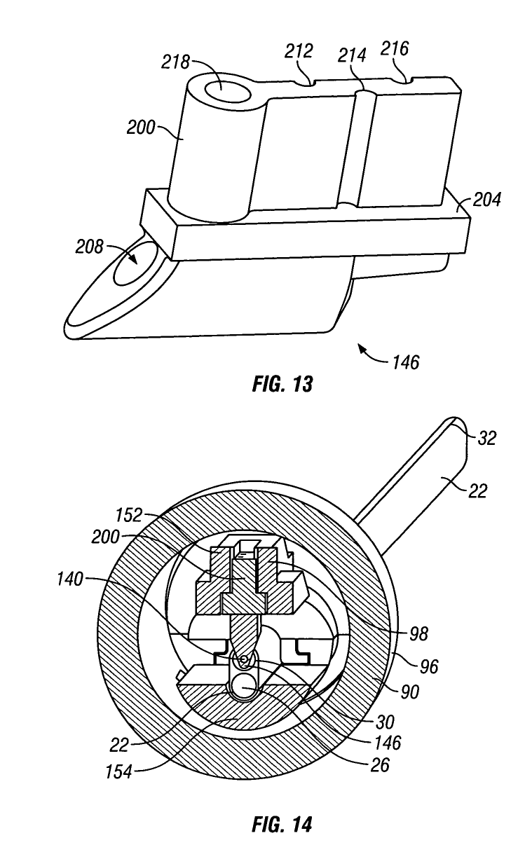

Keel 146 is formed preferably as a separate component from main body 98 to allow more flexibility in the keel design. The size, shape and material of keel 146 will not be limited by the manufacture of main body 98. Keel 146 contains an upper portion 200 that is designed to mate with slot 202 in top 152. A positive stop, shoulder 204 surrounds upper portion 200 and mates with ledge 206 of slot 202 as shown in FIG. 11. Once mated, keel passageway 208 is in proper alignment with passageway 134 extending through top 152. Upper portion 200 does not extend to be flush with outer surface 208 of top 152 forming recess 210. Adhesive channels 212, 214 and 216 extend along the sides of upper portion 200 as shown in FIG. 13. A bore 218 extends through upper portion 200 and into passageway 208 that receives tube 138.

Keel 146 is preferably secured to top 152 with an adhesive. Keel 146 is inserted into slot 202 until shoulder 204 is firmly seated against ledge 206. Once seated passageways 208 and 134 are aligned so that tube 140 may be inserted. Accordingly, tube 138 is inserted into passageway 208 of keel and then into passageway 134. Passageway 134 contains a stop 220 just prior to the area 222 that receives the wire introducer tool. Tube 138 is seated in place once it abuts stop 220. Adhesive is then placed in channels 212, 214 and 216 securing keel 146 to top 152. Adhesive is also placed in bore 218 that extends down to tube 140. Any excess adhesive will pool in recess 210 and not interfere with fitting the various components of guide member 10 together.

The guide member is preferably made of Blends of Polyamides and Polyolefins—preferred, other suitable materials include Polyamides, Liquid Crystal Polymers, Lubrication additives (used in any polymer) including PE micro-powders, Fluoropolymers, silicone based oils, fluoro-ether oils, Molybdenum disulphide, and Polyethylene oxide; Reinforcing additives including nano-clays, graphite, carbon fibres, glass fibres etc., Polyesters, Polyketones, Polyimides, Polysulphones, Polyoxymethylenes, Polyolefins, Cross-linked polyolefins, Compatibilisers based on Polyolefins, including grafted Polyolefins, Ceramics and Metals, for example stainless steel.

The operation of the device will now be described with reference to FIGS. 1, 2, 6 and 14. Once guide wire 14 and guide catheter (not shown) are inserted into the patient, the catheter 12 is inserted with a backloading operation. Guidewire 14 is inserted into distal end 18 of catheter 12 and threaded proximally through guide wire lumen 30 until guide wire tube 138 captures proximal end of guidewire 14 and directs it into passageway 134 and then out of guide member 10 as shown in FIG. 1. This procedure is typically accomplished with the guide member 10 adjacent the guide way distal end. The guide member 10 may be positioned between stop 36 and speed bump 44. This will keep guide member in proper position during the backloading operation as the force of the wire entering the guide member is insufficient to push the guide member proximally over the speed bump 44. Likewise, if the docking arrangement is used, stop 36 will be adjacent distal end of guide member 10 and will be engaged in docking area 42 to hold guide member 10 in place during the backloading operation. As distal shaft 20 enters the patient, guide member 10 will reach the hemostatic valve (not shown). Guide member 10 is not intended to enter the valve and is seated adjacent the valve. Proximal shaft 22 is then moved though guide member 10 seated against the valve. As proximal shaft 22 is advanced, keel 146 engages guide way 32 as shown in FIG. 14. Guideway closing aids 148 and 150 located on either side of keel assist in biasing guideway 32 to its closed position. Angled edges on closing aids 148 and 150 reduce contact with guideway 32 to keep contact between guideway 32 and proximal shaft 22 at a minimum and ensure prompt closing of guideway 32.

Once inserted, the hemostatic valve may be closed down on the catheter shaft distal of guide member 10. Since tube 138 extends into distal shaft 20 sufficiently the valve clamping forces will be felt on tube 138. Apertures 142 on tube distal portion 140 help achieve a more effective seal around catheter shaft and guidewire 14. If a wire change is required, one simply withdraws the guide wire 14 from the guide member 10 as it is seated against the valve and proximal shaft 22 remains in the patient. A new guide wire is then inserted into the catheter through passageway 134 on guide member. If a catheter exchange is required, one simply holds the wire in place and begins moving the proximal shaft 22 proximal though the guide member which is kept at the hemostatic valve. Once stop 36 on proximal shaft 22 is adjacent guide member 10, the remaining portion of the catheter is removed while the guidewire is still held in place. Another catheter may then be backloaded onto the guide wire and introduced into the patient as described above.

While the invention has been particularly shown and described with reference to the preferred embodiments thereof, it will be understood by those skilled in the art that various changes in form and detail may be made there in without departing from the spirit and scope of the invention.

What is claimed is:

1. A catheter and guide wire exchange system comprising:
an elongate flexible catheter shaft having proximal and distal shafts and first and second lumens extending there through, the first lumen being open at the shaft distal end and being sized and shaped to slidably receive a guide wire;
a longitudinal guide way formed in the proximal shaft to enable transverse access to the first lumen through the proximal shaft, the guide way extending along a major portion of the length of the proximal shaft from a location adjacent a proximal end of the proximal shaft to a distal terminal end proximal of a distal end of the proximal shaft, thereby defining an uncut distal segment of the proximal shaft;
a balloon mounted about a distal segment of the distal shaft, the balloon being in fluid communication with the second lumen; and
a guide member mounted on the proximal shaft and having a catheter passageway extending there through for slidably receiving the catheter shaft and a guide wire passageway for slidably receiving the guide wire for merging the guide wire and the catheter by guiding the guide wire transversely through the guide way and into the first lumen and for separating the guide wire and catheter by guiding the guide wire transversely out of the first lumen through said guide way, the guide member including a keel for tracking the guide way, the guide member having an outer member and an inner member, the outer member being selectively rotatably around the inner member

2. A catheter and guidewire system of claim 1 and further including a keel on the inner member of the guide member, the keel having a passageway for receiving the guidewire.

3. A catheter and guidewire system of claim 2 and further including a guidewire passageway on the inner member that is in communitcation with the keel guide wire passageway.
